(19) United States
(12) Patent Application Publication
(10) Pub. No.: US 2021/0163324 A1
(43) Pub. Date: Jun. 3, 2021

(54) RIVER COURSE TREATMENT INTEGRATION SYSTEM

(71) Applicant: JIANGSU EASTHIGH ENVIRONMENTAL HOLDINGS LTD., Jurong (CN)

(72) Inventors: Jiukun HU, Jurong (CN); Xuejie LIU, Jurong (CN); Liangliang SONG, Jurong (CN); Shengqiao MEI, Jurong (CN); Ren REN, Jurong (CN)

(73) Assignee: JIANGSU EASTHIGH ENVIRONMENTAL HOLDINGS LTD., Jurong (CN)

(21) Appl. No.: 16/699,678

(22) Filed: Dec. 1, 2019

(51) Int. Cl. C02F 3/20; C02F 3/32; G01N 33/18; E02B 3/02; G06T 7/11; G06T 7/12; G06T 7/136; H04W 4/80

(52) U.S. Cl. CPC C02F 3/202; C02F 3/327; G01N 33/182; E02B 3/02; G06Q 50/26; G06T 7/12; G06T 7/136; H04W 4/80; G06T 7/11

(57) ABSTRACT

The invention discloses a river course treatment integration system comprising an information collection module, a treatment module, an audio-visual module and a calculation processing module; the information collection module is used for collecting pollution source information on the periphery of a river course, detecting water quality of the river course, and transferring the information to the calculation processing module through a wireless radio communication signal sending module; the treatment module is used for controlling the pollution source of the river course, and carrying out ecological restoration. The invention collects the pollution source information on the periphery of the river course and detects the water quality of the river course by the information collection module, treats the river course by the treatment module, monitors the pollution source information collected by the information collection module using a source monitoring module, and to some extent, can fundamentally treat the river course, and can thoroughly solve the eutrophication development of a water body using a planting unit, a breeding unit, an aeration device and a decentralized sewage treatment station.

The invention belongs to the technical field of river course treatment, and particularly relates to a river course treatment integration system.

A river course treatment integration technology is an engineering measure for controlling and reconstructing a river course; scour and siltation phenomena often occur in natural rivers, which is easy to cause water disaster and hinder the development of water conservancy; in order to meet the need for eliminating dangers and promoting beneficial projects, river courses must be treated by proper measures; the landscape function of a river is restored by projects of guiding, dredging, revetment and the like; the self-cleaning function of a water body is activated by utilizing micro-power in-situ restoration so that the black odor can be eliminated in a short time, and meanwhile, sludge can be reduced. The treatment is divided into an endogenous pollution treatment and an exogenous pollution treatment.

Current river course treatment integration technologies often employ dynamic water exchange, mechanical filtration, chemical disinfection and sterilization and microbial strain stocking technology; the above technologies all can make the river water clear, but do not fundamentally treat the river and cannot thoroughly solve the eutrophication development of the water body; in addition, these technologies are single technologies; the degree of technology integration is not high, thereby limiting the application of the technologies.

Therefore, we propose a river course treatment integration system to solve the problems existing in the prior art, so that the integration degree of the river course treatment is high, and the river course is treated fundamentally.

### SUMMARY

The object of the invention is to provide an alarm receiving system integrated on a smart police vehicle, so as to solve the problem in the above background technologies that the integration degree is not high, that there is no fundamental treatment, and that the problem of eutrophication development of the water body cannot be thoroughly solved.

In order to achieve the above object, the invention employs the following technical solution:

A river course treatment integration system, comprising an information collection module, a treatment module, an audio-visual module and a calculation processing module, wherein the information collection module is used for collecting the pollution source information on the periphery of a river course and detecting the water quality of the river course, and transferring the information to the calculation processing module through a radio communication signal sending module; the treatment module is used for controlling the pollution source of the river course, and carrying out ecological restoration; the calculation processing module is used for analysis according to the river course pollution conditions collected by the information collection module, planning a best-quality river course treatment scheme, and simulating an evolution and development result after the ecological restoration; and the audio-visual module is used for visually feeding back the river course treatment scheme planned by the calculation processing module and various data of the river course treatment.

Preferably, the wireless communication signal sending module comprises a TD-LTE and FDD-LTE type 4G communication device, a GSM 2G communication device and a WiFi communication device; the wireless communication signal sending module is connected to the information collection module.

Preferably, the treatment module comprises a source monitoring module, a planting unit and a breeding unit; the source monitoring module is used for monitoring the pollution source information collected by the information collection module, and the source monitoring module is electrically connected with the calculation processing module; the planting unit is used for introducing submerged plants into the river course and planting vegetation on both sides of the river; the breeding unit is used for breeding fishes and microorganisms in the river course to improve water quality; the breeding unit comprises an aeration device; the aeration device is used for supplying oxygen for the fishes and microorganisms in the river course to improve the liveness of the fishes and microorganisms, thereby increasing the efficiency for improving the water quality of the river course.

Preferably, the aeration device is configured as a membrane type microporous aerator; the membrane type microporous aerator is small in aeration bubble diameter, small in gas-liquid interface diameter, large in gas-liquid interface area and uniform in bubble diffusion without generating pore blockage, and has a strong corrosion resistance; compared with conventional products, a fixed spiral aerator, a diffusion aerator and a perforated pipe aerator, the energy consumption is reduced by 40%, or the sewage treatment amount is increased by 40%, and the aeration tank can run intermittently.

Preferably, the treatment module further comprises a decentralized sewage treatment station; the decentralized sewage treatment station consists of a filter bed, a water distribution assembly and a drainage assembly; when the sewage flows from top to bottom through the gaps of a filter material with rich biofilms and sufficiently contacts with the microorganisms in the biofilms, the pollutants in the sewage are adsorbed by the microorganisms, completely degraded and eventually converted into CO₂ and N₂; therefore, the sewage is purified.

Preferably, the information collection module comprises an information input module; the information input module comprises a keyboard, a mouse and an electronic writing board; the information output interface supports one or a combination of the following interfaces: a universal serial interface, a computer serial interface, a computer parallel interface, a PCMCIA interface, a PS/2 interface, a wireless communication interface and an infrared communication interface.

Preferably, the audio-visual module comprises a digital-to-analog converter, which is used for processing the conversion between digital signals and analog signals; a display, which is used for displaying an image; an image segmentation unit, which is used for processing the fed-in digital image to be displayed in a digital segmentation area on the display; a sound amplifier, which is used for amplifying the sound.

Preferably, the image segmentation unit is a technology and process for dividing an image into a plurality of specific regions with unique properties and proposing a target of interest; it is a key step from image processing to image analysis, and is mainly divided into the following categories: a threshold-based segmentation method, a region-based segmentation method, an edge-based segmentation method and a specific theory-based segmentation method, etc. From a mathematical perspective, image segmentation is a process of dividing a digital image into non-intersecting regions.

Preferably, the calculation processing module comprises a signal receiving module; the signal receiving module is used for receiving a radio signal sent by the wireless communication signal sending module; the signal receiving module comprises a WIFI antenna, an LTE antenna, a radio frequency switch, a frequency divider and a processor, wherein the radio frequency switch comprises a first input port, a second input port and a WIFI signal output port, and the frequency divider comprises a third input port, a first output port and a second output port; the WIFI antenna is connected with the first input port; the LTE antenna is connected with the third input port; the first output port is connected with the second input port; the frequency divider is used for separating WIFI signals from LTE signals in the signals received by the LTE antenna, and outputting the separated WIFI signals and LTE signals through the first output port and the second output port, respectively.

The technical effects and advantages of the invention: compared with the prior technology, the river course treatment integration system has the following advantages:

The invention collects the pollution source information on the periphery of the river course and detects the water quality of the river course by the information collection module, treats the river course by the treatment module, monitors the pollution source information collected by the information collection module by the source monitoring module, and to some extent, can fundamentally treat the river course, and can thoroughly solve the eutrophication development of the water body by the planting unit, the breeding unit, the aeration device and the decentralized sewage treatment station.

### BRIEF DESCRIPTION OF THE DRAWING

FIG. 1 is a block diagram of the river course treatment integration technology system according to the invention.

### DETAILED DESCRIPTION OF THE EMBODIMENT

The technical solutions in the examples of the invention are clearly and completely described hereinbelow. It is obvious that the described example is only part of the examples of the invention, but not all of the examples. The specific example described herein is only used to illustrate the invention and is not used to limit the invention. All other examples obtainable by those of ordinary skill in the technology based on the examples of the invention without doing inventive work are within the protection scope of the invention.

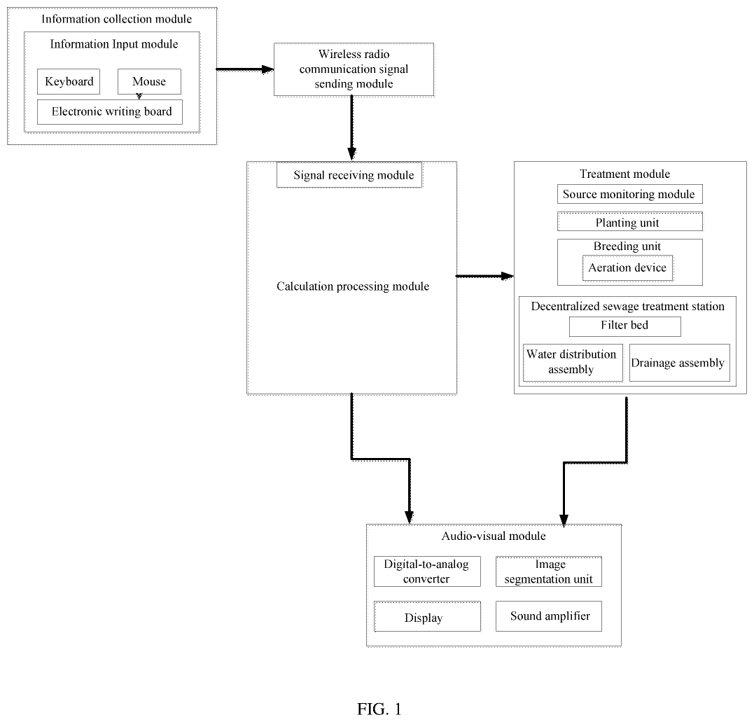

The invention provides a river course treatment integration system as shown in FIG. 1, comprising an information collection module, a treatment module, an audio-visual module and a calculation processing module; the information collection module is used for collecting the pollution source information on the periphery of the river course and detecting the water quality of the river course, and transferring the information to the calculation processing module through a wireless radio communication signal sending module; the treatment module is used for controlling the pollution source of the river course, and carrying out ecological restoration; the calculation processing module is used for analysis according to the river course pollution conditions collected by the information collection module, planning a best-quality river course treatment scheme, and simulating an evolution and development result after the ecological restoration; and the audio-visual module is used for visually feeding back the river course treatment scheme planned by the calculation processing module and various data of the river course treatment.

Preferably, the wireless communication signal sending module comprises a TD-LTE and FDD-LTE type 4G communication device, a GSM 2G communication device and a WiFi communication device; the wireless communication signal sending module is connected to the information collection module.

Preferably, the treatment module comprises a source monitoring module, a planting unit and a breeding unit; the source monitoring module is used for monitoring the pollution source information collected by the information collection module, and the source monitoring module is electrically connected with the calculation processing module; the planting unit is used for introducing submerged plants into the river course and planting vegetation on both sides of the river; the breeding unit is used for breeding fishes and microorganisms in the river course to improve water quality; the breeding unit comprises an aeration device; the aeration device is used for supplying oxygen for fishes and microorganisms in the river course to improve the liveness of the fishes and microorganisms, thereby increasing the water quality improvement efficiency of the river course.

The aeration device is configured as a membrane type microporous aerator; the membrane type microporous aerator is small in aeration bubble diameter, small in gas-liquid interface diameter, large in gas-liquid interface area and uniform in bubble diffusion without generating pore blockage, and has a strong corrosion resistance; compared with conventional products, a fixed spiral aerator, a diffusion aerator and a perforated pipe aerator, the energy consumption is reduced by 40%, or the sewage treatment amount is increased by 40%, and the aeration tank can run intermittently.

The treatment module further comprises a decentralized sewage treatment station; the decentralized sewage treatment station consists of a filter bed, a water distribution assembly and a drainage assembly; when the sewage flows from top to bottom through the gaps of a filter material with rich biofilms and sufficiently contacts with the microorganisms in the biofilms, the pollutants in the sewage are adsorbed by the microorganisms, completely degraded and eventually converted into CO₂ and N₂; therefore, the sewage is purified.

The information collection module comprises an information input module; the information input module comprises a keyboard, a mouse and an electronic writing board; the information output interface supports one or a combination of the following interfaces: a universal serial interface, a computer serial interface, a computer parallel interface, a PCMCIA interface, a PS/2 interface, a wireless communication interface and an infrared communication interface.

The audio-visual module comprises a digital-to-analog converter, which is used for processing the conversion between digital signals and analog signals; a display, which is used for displaying the image; an image segmentation unit, which is used for processing the fed-in digital image to be displayed in a digital segmentation area on the display; a sound amplifier, which is used for amplifying the sound.

The image segmentation unit is a technology and process for dividing an image into a plurality of specific regions with unique properties and proposing a target of interest; it is a key step from image processing to image analysis, and is mainly divided into the following categories: a threshold-based segmentation method, a region-based segmentation method, an edge-based segmentation method and a specific theory-based segmentation method, etc. From a mathematical perspective, image segmentation is a process of dividing a digital image into non-intersecting regions.

The calculation processing module comprises a signal receiving module; the signal receiving module is used for receiving a radio signal sent by the wireless communication signal sending module; the signal receiving module comprises a WIFI antenna, an LTE antenna, a radio frequency switch, a frequency divider and a processor, wherein the radio frequency switch comprises a first input port, a second input port and a WIFI signal output port, and the frequency divider comprises a third input port, a first output port and a second output port; the WIFI antenna is connected with the first input port; the LTE antenna is connected with the third input port; the first output port is connected with the second input port; the frequency divider is used for separating WIFI signals from LTE signals in the signals received by the LTE antenna, and outputting the separated WIFI signals and LTE signals through the first output port and the second output port, respectively.

Working principle: the invention collects the pollution source information on the periphery of the river course and detects the water quality of the river course by the information collection module, treats the river course by the treatment module, monitors the pollution source information collected by the information collection module by the source monitoring module, and to some extent, can fundamentally treat the river course, and can thoroughly solve the eutrophication development of the water body by the planting unit, the breeding unit, the aeration device and the decentralized sewage treatment station.

Finally, it should be noted that the above example is a preferred embodiment of the invention, and is not used to limit the invention. Although the invention is described with detail with reference to the above example, those of ordinary skill in this technology still can modify the technical solutions disclosed in the above various examples, or equivalently replace a part of the technical features therein. Any modification, equivalent replacement, improvement and the like that are made without departing from the spirit and principle of the invention should be included in the protection scope of the invention.

What is claimed is:

1. A river course treatment integration system, comprising an information collection module, a treatment module, an audio-visual module and a calculation processing module, characterized in that the information collection module is used for collecting pollution source information on the periphery of a river course and detecting water quality of the river course, and transferring the information to the calculation processing module through a wireless radio communication signal sending module; the treatment module is used for controlling the pollution source of the river course, and carrying out ecological restoration; the calculation processing module is used for analysis according to the river course pollution condition collected by the information collection module, planning a best-quality river course treatment scheme, and simulating an evolution and development result after the ecological restoration; and the audio-visual module is used for visually feeding back the river course treatment scheme planned by the calculation processing module and various data of the river course treatment.

2. The river course treatment integration system according to claim 1, characterized in that the wireless communication signal sending module comprises a TD-LTE and FDD-LTE type 4G communication device, a GSM 2G communication device and a WiFi communication device; the wireless communication signal sending module is connected to the information collection module.

3. The river course treatment integration system according to claim 1, characterized in that the treatment module comprises a source monitoring module, a planting unit and a breeding unit; the source monitoring module is used for monitoring the pollution source information collected by the information collection module, and the source monitoring module is electrically connected with the calculation processing module; the planting unit is used for introducing submerged plants into the river course and planting vegetation on both sides of the river; the breeding unit is used for breeding fishes and microorganisms in the river course to improve water quality; the breeding unit comprises an aeration device, the aeration device being used for supplying oxygen for fishes and microorganisms in the river course to improve the liveness of the fishes and microorganisms, thereby increasing the efficiency for improving the water quality of the river course.

4. The river course treatment integration system according to claim 3, characterized in that the aeration device is configured as a membrane type microporous aerator; the membrane type microporous aerator is small in aeration bubble diameter, small in gas-liquid interface diameter, large in gas-liquid interface area and uniform in bubble diffusion without generating pore blockage, and has a strong corrosion resistance; compared with conventional products, a fixed spiral aerator, a diffusion aerator and a perforated pipe aerator, the energy consumption is reduced by 40%, or the sewage treatment amount is increased by 40%, and the aeration tank can run intermittently.

5. The river course treatment integration system according to claim 3, characterized in that the treatment module further comprises a decentralized sewage treatment station; the decentralized sewage treatment station consists of a filter bed, a water distribution assembly and a drainage assembly; when sewage flows from top to bottom through the gaps of a filter material with rich biofilms and sufficiently contacts with the microorganisms in the biofilms, the pollutants in the sewage are absorbed by the microorganisms, completely degraded and eventually converted into CO₂ and N₂, so that the sewage is purified.

6. The river course treatment integration system according to claim 1, characterized in that the information collection module comprises an information input module; the information input module comprises a keyboard, a mouse and an electronic writing board; the information output interface supports one or a combination of the following interfaces: a universal serial interface, a computer serial interface, a computer parallel interface, a PCMCIA interface, a PS/2 interface, a wireless communication interface and an infrared communication interface.

7. The river course treatment integration system according to claim 1, characterized in that the audio-visual module comprises a digital-to-analog converter, which is used for processing the conversion between digital signals and analog signals; a display, which is used for displaying the image; an image segmentation unit, which is used for processing the fed-in digital image to be displayed in a digital segmentation area on the display; a sound amplifier, which is used for amplifying the sound.

8. The river course treatment integration system according to claim 6, characterized in that the image segmentation unit is a technology and process for dividing an image into a plurality of specific regions with unique properties and proposing a target of interest; it is a key step from image processing to image analysis, and is mainly divided into the following categories: a threshold-based segmentation method, a region-based segmentation method, an edge-based segmentation method and a specific theory-based segmentation method, etc. From a mathematical perspective, image segmentation is a process of dividing a digital image into non-intersecting regions.

9. The river course treatment integration system according to claim 1, characterized in that the calculation processing module comprises a signal receiving module; the signal receiving module is used for receiving a wireless radio signal sent by the wireless communication signal sending module; the signal receiving module comprises a WIFI antenna, a LTE antenna, a radio frequency switch, a frequency divider and a processor; the radio frequency switch comprises a first input port, a second input port and a WIFI signal output port; the frequency divider comprises a third input port, a first output port and a second output port; the WIFI antenna is connected with the first input port; the LTE antenna is connected with the third input port; the first output port is connected with the second input port; the frequency divider is used for separating WIFI signals from LTE signals in the signals received by the LTE antenna, and outputting the separated WIFI signals and LTE signals through the first output port and the second output port, respectively.
